(19) United States
(12) Patent Application Publication
(10) Pub. No.: US 2007/0083229 A1
(43) Pub. Date: Apr. 12, 2007

(54) Method and device for cavity obliteration

(75) Inventor: Harvey L Deutsch, Los Angeles, CA (US)

Correspondence Address: SHELDON MAK ROSE & ANDERSON PC, 225 SOUTH LAKE AVENUE, 9TH FLOOR, PASADENA, CA, 91101, US

(73) Assignee: The Catheter Exchange, Inc.

(21) Appl. No.: 10/548,857

(22) PCT Filed: Jun. 17, 2005
(86) PCT No.: PCT/US05/21615

§ 371 Date: Sep. 9, 2005

Related U.S. Application Data
(60) Provisional application No. 60/580,784, filed on Jun. 18, 2004.

(51) Int. Cl. A61B 17/08

(52) U.S. Cl. 606/213; 606/151

(57) ABSTRACT

A device for the obliteration of an aberrant space or cavity comprising a disk of material comprising a first side, an opposing second side, and a perimeter circumferentially surrounding the first side and the second side. A method for the obliteration of an aberrant space or cavity comprises an open end and a closed end comprising, a) selecting an aberrant space or cavity that is suitable for obliteration by the method; b) creating an opening in the closed end of the aberrant space or cavity; c) providing a device for the obliteration of an aberrant space or cavity; and d) deploying the device through the opening created in the closed end of the aberrant space or cavity to substantially seal the open end of the aberrant space or cavity, thereby obliterating the aberrant space or cavity.

### CROSS-REFERENCE TO RELATED APPLICATIONS

The present Application is a United States national phase application of International Patent Application No. PCT/US05/21615, titled “Method and Device for Cavity Obliteration,” filed Jun. 17, 2005, which claims the benefit of United States Provisional Patent Application No. 60/580,784 titled “Percutaneous Cavity Obliteration Device,” filed Jun. 18, 2004, the contents of which are incorporated in this disclosure by reference in their entirety.

### BACKGROUND

There are a number of human diseases and conditions that include the existence of an aberrant space or cavity, such as, for example, a hernia sac of an inguinal hernia, and for which treatment of the disease or condition involves obliteration of the space or cavity. Many treatments have been developed for the obliteration of the hernia sac of an inguinal hernia, including both open surgical and laparoscopic procedures using polypropylene (Marlex®) patches. These patches induce a fibrotic reaction leading to obliteration of the hernia sac. Disadvantageously, however, the polypropylene surface of the patch must be separated from the intraperitoneal contents during the repair because polypropylene in contact with intraperitoneal structures can induce intraperitoneal adhesions causing post-operative bowel obstructions. Separation of the polypropylene surface of the patch from the intraperitoneal contents is generally accomplished by tacking a layer of peritoneum over the polypropylene patch used to cover the entrance into the hernia sac. Alternatively, a composite patch combining both a polytetrafluoroethylene (PTFE) layer and polypropylene layer is used. The composite patch is positioned so that the PTFE layer faces the peritoneal cavity because PTFE does not induce intraperitoneal adhesions, and because the PTFE layer provides a barrier to the fibrotic response caused by polypropylene within the hernia sac.

There are, however, a number of disadvantages to both currently used laparoscopic and open surgical approaches for the repair of inguinal hernias. For example, open surgery involves a moderately sized skin incision that carries with it the risks of wound dehiscence, infection, post-operative pain, hernia recurrence, and a significant recuperative period. By contrast, laparoscopic techniques require general anesthesia, at least three abdominal skin punctures, distention of the peritoneal cavity with carbon dioxide gas, possible longer operative times, and an increased potential for bowel and neurovascular injuries. Therefore, there remains a need for a new method for the obliteration of an aberrant space or cavity, such as the hernia sac of an inguinal hernia.

### SUMMARY

According to one embodiment of the present invention, there is provided a device for the obliteration of an aberrant space or cavity comprising a disk of material comprising a first side, an opposing second side, and a perimeter circumferentially surrounding the first side and the second side. In one embodiment, the material comprises polypropylene or polytetrafluoroethylene or both polypropylene or polytetrafluoroethylene. In another embodiment, the device further comprises a frame forming the perimeter, where the material forming the first side and the second side are stretched over the frame and attached to the frame. In one embodiment, the frame comprises a shape selected from the group consisting of substantially round, oval, square, rectangular, kidney shaped, and clover leaf shaped with a plurality of leaves. In another embodiment, the frame comprises a shaped metal alloy. In a preferred embodiment, the device further comprises a central layer of compressible material between the first side and the second side, and within the perimeter. In one embodiment, the central layer comprises a biocompatible, elastic memory foam whose final shape is attained after the application of heat provided by one or more than one resistive heating element embedded within the central layer. In another embodiment, the central layer comprises a cold hibernated elastic memory, polyurethane-based foam or a shaped memory polymer. In one embodiment, the first side of the device, the second side of the device, or both the first side and the second side of the device are covered, at least in part, by a material that inhibits a fibrotic reaction in a human. In another embodiment, the first side of the device, the second side of the device, or both the first side and the second side of the device are covered, at least in part, by a material that promotes a fibrotic reaction in a human.

According to one embodiment of the present invention, the device further comprises one or more than one attachment portion that is configured to attach one side of the device to a surface or structure adjacent to or within the aberrant space or cavity to be obliterated thereby immobilizing the device in position. In one embodiment, the attachment portion comprises a main section comprising a first end, and a second end joined to the first surface of the device. In another embodiment, the main section comprises a self-expanding stent comprising a wire comprising a plurality of angles to form a cylindrical shape. In another embodiment, the first end of the main section comprises a plurality of connectors configured to join the device to a surface or structure adjacent to or within the aberrant space or cavity to be obliterated. In a preferred embodiment, the connectors comprise barbs comprises sharp tips directed toward the first surface of the device.

According to one embodiment of the present invention, the device further comprises a plurality of peripheral tethering sutures, each peripheral tethering suture comprises a free first end, and comprises a second end joined to the first surface of the device. In one embodiment, the plurality of peripheral tethering sutures is between 2 and about 10 tethering sutures. In another embodiment, the plurality of peripheral tethering sutures is between about 4 and about 6 tethering sutures. In one embodiment, the second end of the one or more than one peripheral tethering sutures comprises a plurality of secondary sutures joining the second end peripheral tethering suture to the first surface. In another embodiment, the plurality of secondary sutures is between 2 and about 10 secondary sutures. In one embodiment, the device further comprises a central tethering suture or a central stabilization wire comprising a free first end, and a second end joined to the first surface of the device.

According to one embodiment of the present invention, the device further comprises a plurality of wires, each wire comprises a free first end, and a second end joined to the first surface of the device, where the first end of each wire bends between approximately 100° and 180° and comprises a sharp tip directed toward the first surface of the device. In one embodiment, the plurality of wires is between about 2 and about 10 wires. In another embodiment, the plurality of wires is between about 4 and about 6 wires. In a preferred embodiment, the device further comprises a material skirt surrounding the second ends of the wires and extending toward the first free end of the wires, but not covering the first free ends of the wires.

According to one embodiment of the present invention, the device further comprises a mass of material attached to the first surface of the device, where the mass of material comprises one or more than one substance or composition known to promote a fibrotic reaction in a human.

According to one embodiment of the present invention, the device further comprises a cold hibernated elastic memory, polyurethane-based foam. In one embodiment, the device further comprises one or more than one resistive heating element.

According to one embodiment of the present invention, the device consists essentially of biocompatible, elastic memory foam and one or more than one resistive heating element embedded within the disk. According to one embodiment of the present invention, the device consists of biocompatible, elastic memory foam and one or more than one resistive heating element embedded within the disk.

According to one embodiment of the present invention, the first side of the device is covered, partially or totally, with a material that promotes a fibrotic reaction in a human.

According to one embodiment of the present invention, the second side of the device is covered, partially or totally, with a material that inhibits a fibrotic reaction in a human.

According to one embodiment of the present invention, the device further comprises one or more than one inflation channel between the first side and the second side. In one embodiment, the device further comprises a connector in communication with the one or more than one inflation channel for interfacing with an inflation mechanism.

According to one embodiment of the present invention, the device further comprises a frame, where the frame comprises a plurality of peripherally radiating members comprises a first end and a second end, where the first end of each radiating member is joined at a central connector, and where the second end of one or more than one of the radiating members comprises a clip to attach the radiating member to a surface or structure adjacent to or within the aberrant space or cavity to be obliterated thereby immobilizing the device in position. In one embodiment, the device further comprises an actuating mechanism for approximating the second ends of the radiating members. In another embodiment, the frame comprises wire comprises a shaped metal alloy or a shaped memory alloy. In another embodiment, each clip comprises a plurality of arms comprises a first end and a second end. In a preferred embodiment, the first ends of each arm are joined to an attachment line. In another preferred embodiment, the second end of each arm comprises one or more than one gripping tip. In one embodiment, each clip further comprises a tubular structure surrounding the first end and attached to the frame.

According to one embodiment of the present invention, there is provided a method for the obliteration of an aberrant space or cavity comprises an open end and a closed end. The method comprises, a) selecting an aberrant space or cavity that is suitable for obliteration by the method; b) creating an opening in the closed end of the aberrant space or cavity; c) providing a device for the obliteration of an aberrant space or cavity; and d) deploying the device through the opening created in the closed end of the aberrant space or cavity to substantially seal the open end of the aberrant space or cavity, thereby obliterating the aberrant space or cavity. In one embodiment, the aberrant space or cavity obliterated by the method is within a living organism. In another embodiment, selecting the aberrant space or cavity comprises diagnosing the existence of an aberrant space or cavity in a human using a technique selected from the group consisting of CT scan, herniography, history, MRI and physical examination. In another embodiment, the aberrant space or cavity obliterated by the method is a hernia sac of an inguinal hernia in a patient, and where the open end of the hernia sac is in communication with the peritoneal cavity of the patient. In another embodiment, the opening in the closed end is created using a percutaneous transcatheter approach. In another embodiment, the device is deployed through the opening created in the closed end. In one embodiment, the method further comprises making a puncture incision in the skin of the lower abdomen of the patient. In another embodiment, the method further comprises inflating the peritoneal cavity with a gas to distend the hernia sac. In a preferred embodiment, creating an opening in the closed end of the hernia sac comprises puncturing the hernia sac with a needle. In another embodiment, the method further comprises advancing a guidewire through the opening in the closed end of the hernia sac, through the hernia sac, and through the open end of the hernia sac into the peritoneal cavity. In another embodiment, the method further comprises removing the needle and advancing an introducer catheter with a central dilator over the guidewire, through the opening in the closed end of the hernia sac, through the hernia sac, and through the open end of the hernia sac into the peritoneal cavity. In another embodiment, deploying the device comprises advancing the device through the introducer catheter. In another embodiment, deploying the device comprises bringing the perimeter of the device toward the center of the device. In another embodiment, deploying the device comprises rolling the device. In another embodiment, deploying the device comprises attaching the device to a pusher rod.

In a preferred embodiment, the device provided is a device according to the present invention.

### FIGURES

These and other features, aspects and advantages of the present invention will become better understood with regard to the following description, appended claims, and accompanying figures which depict various views and embodiments of the device, and some of the steps in certain embodiments of the method of the present invention, where:

FIG. 1 is a perspective view of the first side of one embodiment of the device for the obliteration of an aberrant space or cavity according to the present invention;

FIG. 2 is a perspective view of the second side of the embodiment of the device shown in FIG. 1;

FIG. 3 is a perspective view of the first side of one embodiment of the device for the obliteration of an aberrant space or cavity according to the present invention, comprising a frame comprising a clover leaf shape with six leaves;

FIG. 4 is a perspective view of the first side of one embodiment of the device for the obliteration of an aberrant space or cavity comprising a central layer of compressible material between the first side and the second side, and within the perimeter;

FIG. 5 is a perspective view of the first side of one embodiment of the device for the obliteration of an aberrant space or cavity comprising an attachment portion joined to the first side;

FIG. 6 is a lateral perspective view of the embodiment of the device shown in FIG. 5;

FIG. 7 is a partial perspective view of the first side of another embodiment of the device for the obliteration of an aberrant space or cavity;

FIG. 8 is a partial lateral perspective view of the embodiment of the device shown in FIG. 7;

FIG. 9 is a partial lateral perspective view of another embodiment of the device;

FIG. 10 is a perspective view of the first side of another embodiment of the device for the obliteration of an aberrant space or cavity;

FIG. 11 is a lateral perspective view of the embodiment of the device shown in FIG. 10;

FIG. 12 is a lateral perspective view of the first side of another embodiment of the device for the obliteration of an aberrant space or cavity;

FIG. 13, FIG. 14 and FIG. 15 are lateral perspective views of three other embodiments of the device for the obliteration of an aberrant space or cavity, consisting essentially of biocompatible, elastic memory foam and one or more than one resistive heating element;

FIG. 16 is a lateral perspective view of the first side of another embodiment of the device for the obliteration of an aberrant space or cavity;

FIG. 17 is an exploded, lateral perspective view of the disk portion of another embodiment of the device for the obliteration of an aberrant space or cavity;

FIG. 18, FIG. 19 and FIG. 20 are top perspective views of the second side of three embodiments of the device for the obliteration of an aberrant space or cavity according to the present invention;

FIG. 21 is a lateral perspective view of another embodiment of the device for the obliteration of an aberrant space or cavity in a pre-deployment configuration;

FIG. 22 is a lateral perspective view of the embodiment of the device shown in FIG. 21 in a post-deployment configuration; and

FIG. 23 is a lateral perspective view of the frame portion of the device shown in FIG. 22 in a post-deployment configuration;

FIG. 24, FIG. 25 and FIG. 26 are close-up, lateral perspective views of one embodiment of a clip suitable for incorporation into the embodiment shown in FIG. 21, FIG. 22 and FIG. 23;

FIG. 27 shows a partial cross-sectional view of an aberrant space or cavity comprising an open end and a closed end, suitable for obliteration according to a method of the present invention; and

FIG. 28 through FIG. 36 show partial cross-sectional views of various steps of some embodiments of the method of the present invention for the obliteration of an aberrant space or cavity comprising an open end and a closed end.

### DESCRIPTION

According to one embodiment of the present invention, there is provided a device for the obliteration of an aberrant space or cavity. In a preferred embodiment, the aberrant space or cavity is within a living organism, such as within a human. In another preferred embodiment, the aberrant space or cavity is a hernia sac of an inguinal hernia. According to another embodiment of the present invention, there is provided a method for the obliteration of an aberrant space or cavity comprising an open end and a closed end. In a preferred embodiment, the aberrant space or cavity obliterated by the method is within a living organism, such as within a human. In another preferred embodiment, the aberrant space or cavity obliterated by the method is a hernia sac of an inguinal hernia. In one embodiment, the method comprises providing a device according to the present invention. In another embodiment, the method comprises deploying a device through the opening created in the closed end of the aberrant space or cavity.

As used in this disclosure, the term “comprise” and variations of the term, such as “comprising” and “comprises,” are not intended to exclude other additives, components, integers or steps.

As used in this disclosure, the term “closed end of the aberrant space or cavity” means any position on the wall of the aberrant space or cavity other than through the open end of the aberrant space or cavity.

As used in this disclosure, the term “obliterate” means to substantially seal the open end of the aberrant space or cavity.

All dimensions specified in this disclosure are by way of example only and are not intended to be limiting. Further, the proportions shown in these Figures are not necessarily to scale. As will be understood by those with skill in the art with reference to this disclosure, the actual dimensions of any device or part of a device disclosed in this disclosure will be determined by its intended use.

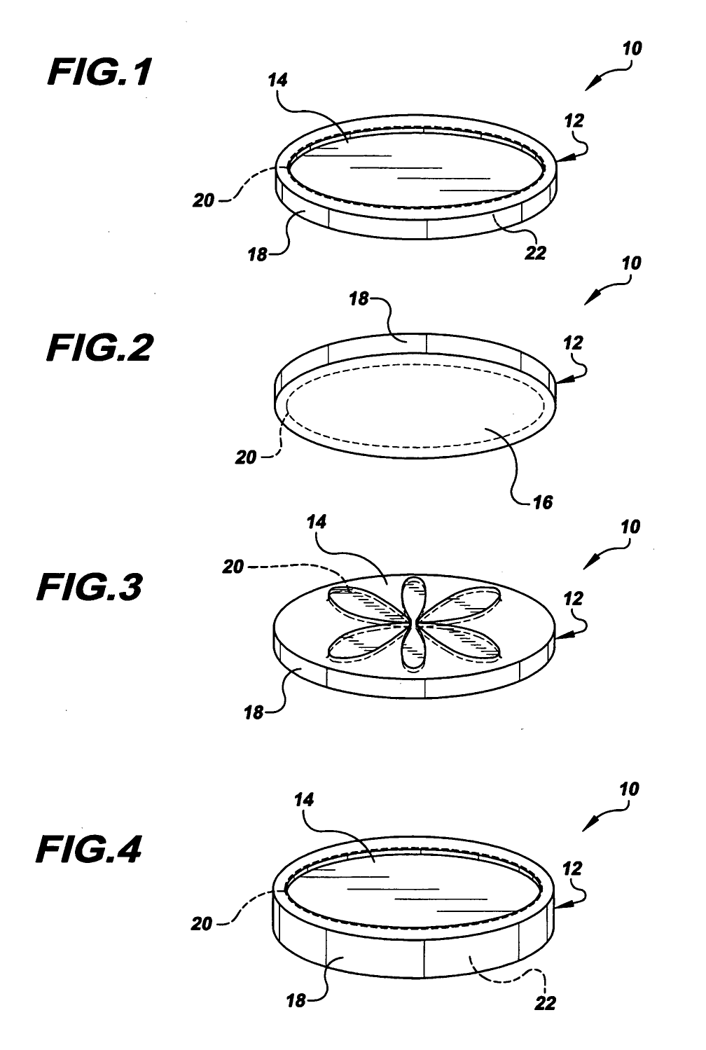

In one embodiment, the present invention is a device for the obliteration of an aberrant space or cavity. In a preferred embodiment, the aberrant space or cavity is within a living organism, such as within a human. In another preferred embodiment, the aberrant space or cavity is a hernia sac of an inguinal hernia. Referring now to FIG. 1 through FIG. 4, there is shown a perspective view of the first side of one embodiment of the device 10 for the obliteration of an aberrant space or cavity according to the present invention (FIG. 1); a perspective view of the second side of the embodiment of the device 10 shown in FIG. 1 (FIG. 2); a perspective view of the first side of one embodiment of the device 10 for the obliteration of an aberrant space or cavity according to the present invention, comprising a frame 20 comprising a clover leaf shape (FIG. 3); and a perspective view of the first side of one embodiment of the device for the obliteration of an aberrant space or cavity comprising a central layer 22 of compressible material between the first side 14 and the second side 16, and within the perimeter 18 (FIG. 4). As can be seen, in one embodiment the device 10 comprises a disk 12 of material comprising a first side 14, an opposing second side 16, and a perimeter 18 circumferentially surrounding the first side 14 and the second side 16. The disk 12 can comprise a single uniform material or composition throughout, such as, for example, a cold hibernated elastic memory (CHEM), reticulated foam, such as a polyurethane-based foam. In a preferred embodiment, however, the disk 12 comprises a frame 20 forming the perimeter 18, with material stretched over the frame 20 forming the first side 14 and the second side 16. The material forming the first side 14 and the second side 16 can be attached to the frame 20 by sutures, an adhesive or by other suitable means, as will be understood by those with skill in the art with reference to this disclosure. In one embodiment, the frame 20 is substantially round, as shown in FIG. 1 and FIG. 2, however, the frame 20 can be any shape suitable for the intended use, as will be understood by those with skill in the art with reference to this disclosure. For example, the frame 20 can be substantially oval, square, rectangular or kidney shaped, or can be another shape, depending on the intended use. In a preferred embodiment, the frame 20 has a clover leaf shape with a plurality of leaves, such as two leaves, three leaves, four leaves, or more than four leaves. In a particularly preferred embodiment, the frame 20 has a clover leaf shape with six leaves, as shown in FIG. 3.

The material forming device 10, including the first side 14, the second side 16, and the frame 20 if present, can be any material suitable for the intended use, as will be understood by those with skill in the art with reference to this disclosure. In one embodiment, the first side 14 and the second side 16 comprise material selected from the group consisting of polypropylene, polytetrafluoroethylene (PTFE) graft material and silicone rubber. In another embodiment, the frame 20 comprises wire, such as a shaped metal alloy or a shaped memory alloy. In a preferred embodiment, the metal alloy is selected from the group consisting of a nitinol and Elgiloy®(RMO, Denver, Colo., US). In a preferred embodiment, when the device 10 is used to obliterate an aberrant space or cavity within a human, the frame 20 comprises wire of a biocompatible material.

In another embodiment, as can be seen in FIG. 4, the device 10 comprises a central layer 22 of compressible material between the first side 14 and the second side 16, and within the perimeter 18. In this embodiment, where the device 10 comprises a central layer 22, a frame can also be present but need not be present. In a preferred embodiment, when the device 10 is used to obliterate an aberrant space or cavity within a human, the central layer 22 comprises a biocompatible material. In a particularly preferred embodiment, the central layer 22 comprises a highly compressible material, such as a reticulated foam. In one embodiment, the reticulated foam is a polyurethane foam. In a particularly preferred embodiment, the central layer 22 comprises a biocompatible, elastic memory foam whose final shape is attained after the application of heat provided by one or more than one resistive heating element embedded within the central layer 22. In a preferred embodiment, the foam is a cold hibernated elastic memory, polyurethane-based foam, such as used for endovascular interventions, as will be understood by one of ordinary skill in the art with reference to this disclosure.

In another embodiment, the first side 14 of the device 10, the second side 16 of the device 10, or both the first side 14 and the second side 16 of the device 10 are covered, at least in part, by a material intended to either inhibit or to promote a fibrotic reaction in a human. For example, when both the first side 14 of the device 10 and the second side 16 of the device 10 comprise a material that promotes a fibrotic reaction in a human, such as polypropylene, one of the sides can be covered, partially or totally, with a material that inhibits a fibrotic reaction in a human, such as a material selected from the group consisting of a hydrophilic material, a biocompatible hydrogel, PTFE and a sodium hyaluronate and carboxy-methylcellulose-based material such as Seprafilm®(Genzyme Corporation Framingham, Mass., US). Similarly, when both the first side 14 of the device 10 and the second side 16 of the device 10 comprise a material which inhibits a fibrotic reaction in a human, such as PTFE, one of the sides can be covered, partially or totally, with a material which promotes a fibrotic reaction in a human, such as polypropylene or polyglycolic acid. For example, when the device 10 is used in a human to repair an inguinal hernia, the side comprising material which promotes a fibrotic reaction in a human can be positioned facing the hernia sac, and the side comprising material which inhibits a fibrotic reaction in a human can be positioned facing the intraperitoneal contents.

The dimensions of the device 10 are determined by the intended use, as will be understood by those with skill in the art with reference to this disclosure. By way of example only, the maximum expanded diameter of the device 10 is between about 5 cm and 7 cm when used to obliterate a typical hernia sac of an inguinal hernia. When a central layer 22 is present, as shown in FIG. 4, the device 10 preferably has a maximum expanded thickness of less than about 5 mm when used to obliterate a hernia sac of an inguinal hernia. In one embodiment, the device 10 has a maximum expanded thickness of between about 1 mm and 5 mm when used to obliterate a hernia sac of an inguinal hernia. In one embodiment, the device 10 has a maximum expanded thickness of between about 2 mm and 3 mm when used to obliterate a hernia sac of an inguinal hernia. As stated above, however, the device 10 can have other dimensions as needed for its intended use. Further, when used to obliterate a hernia sac of an inguinal hernia according to a method of the present invention, the device 10 is preferably highly compressible, allowing it to be percutaneously introduced via a 12-14 F catheter.

In a preferred embodiment, the device 10 further comprises one or more than one attachment portion that is configured to attach the device 10 to a surface or structure adjacent to or within the aberrant space or cavity to be obliterated, such as to the peritoneal surface at the entry site into the hernia sac of an inguinal hernia, thereby immobilizing the device 10 in position. In a preferred embodiment, when the device 10 is used to obliterate the hernia sac of an inguinal hernia, the attachment portion fixes the device 10 in a position covering the junction of the peritoneal cavity and the hernia sac, thereby inducing a fibrotic reaction within the hernia sac and obliterating the hernia sac. The disk 12 portion of the device 10 isolates the intraperitoneal contents from the fibrotic reaction taking place in the hernia sac.

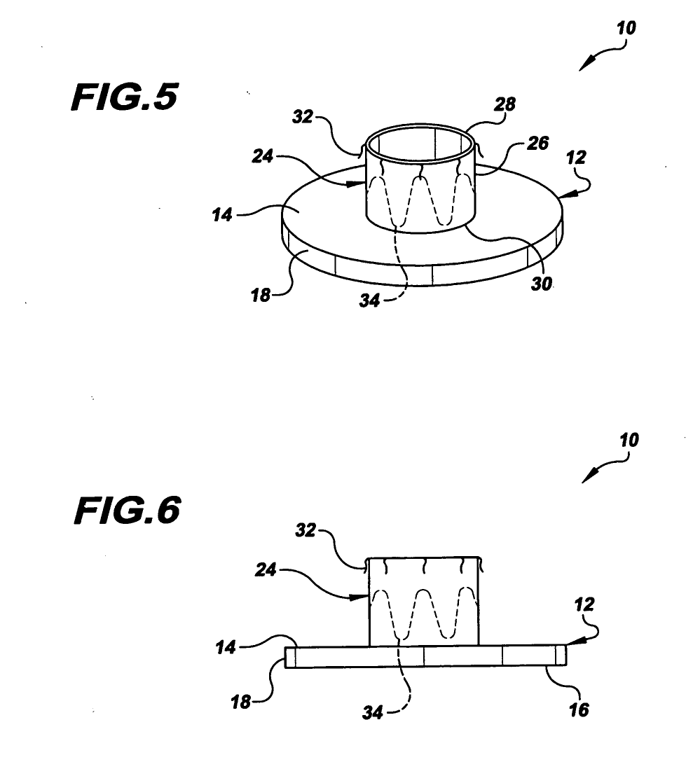

Referring now to FIG. 5 and FIG. 6, there is shown a perspective view of the first side 14 of one embodiment of the device 10 for the obliteration of an aberrant space or cavity comprising an attachment portion 24 joined to the first side 14 (FIG. 5). A lateral perspective view of the embodiment of the device 10 is shown in FIG. 5 (FIG. 6). As can be seen, in one embodiment, the attachment portion 24 comprises a main section 26 comprising a first end 28 and a second end 30. The first end 28 of the main section 26 comprises a plurality of connectors 32 configured to join the device 10 to a surface or structure adjacent to or within the aberrant space or cavity to be obliterated, such as to the peritoneal surface at the entry site into the hernia sac, thereby immobilizing the device 10 in position. The second end 30 of the main section 26 is joined to the first surface of the device 10 using adhesive, sutures, wire loops or as otherwise appropriate, as will be understood by one of ordinary skill in the art with reference to this disclosure. In one embodiment, the main section 26 is solid. In another embodiment, the main section 26 is hollow. In one embodiment, the main section 26 is substantially cylindrical, though any other shape can be used as appropriate for the intended use as will be understood by those with skill in the art with reference to this disclosure. In a preferred embodiment, the main section 26 comprises a self-expanding stent comprising a wire 34 comprising a plurality of angles to form a cylindrical shape, and covered by polypropylene mesh sutured to the wire 34 using a biocompatible resorbable or nonresorbable suture material, such as, for example, Vicryl®(Johnson & Johnson Corp., New Brunswick, N.J., US), similar to biliary stents, although other stent designs are also suitable depending on the intended use, as will be understood by one of ordinary skill in the art with reference to this disclosure. When the device 10 is used to obliterate the hernia sac of an inguinal hernia, the main section 26 will typically have an axial length of between about 0.5 cm and 2 cm and a diameter of between about 2 cm and 3 cm. When the device 10 is used to obliterate the hernia sac of an inguinal hernia, and the main section 26 comprises wire 34, the wire 34 will typically have a diameter of between about 0.15 mm and 0.35 mm. In a preferred embodiment, the connectors 32 comprise barbs comprising sharp tips directed toward the first surface of the device 10. When the device 10 comprises connectors 32 comprising barbs as shown in FIG. 5 and FIG. 6, the barbs join the device 10 to a surface or structure adjacent to the aberrant space or cavity to be obliterated, such as to the peritoneal surface at the entry site into the hernia sac of an inguinal hernia, by penetrating into the peritoneal surface to a depth of approximately 1 mm, thereby immobilizing the device 10 at its deployment site.

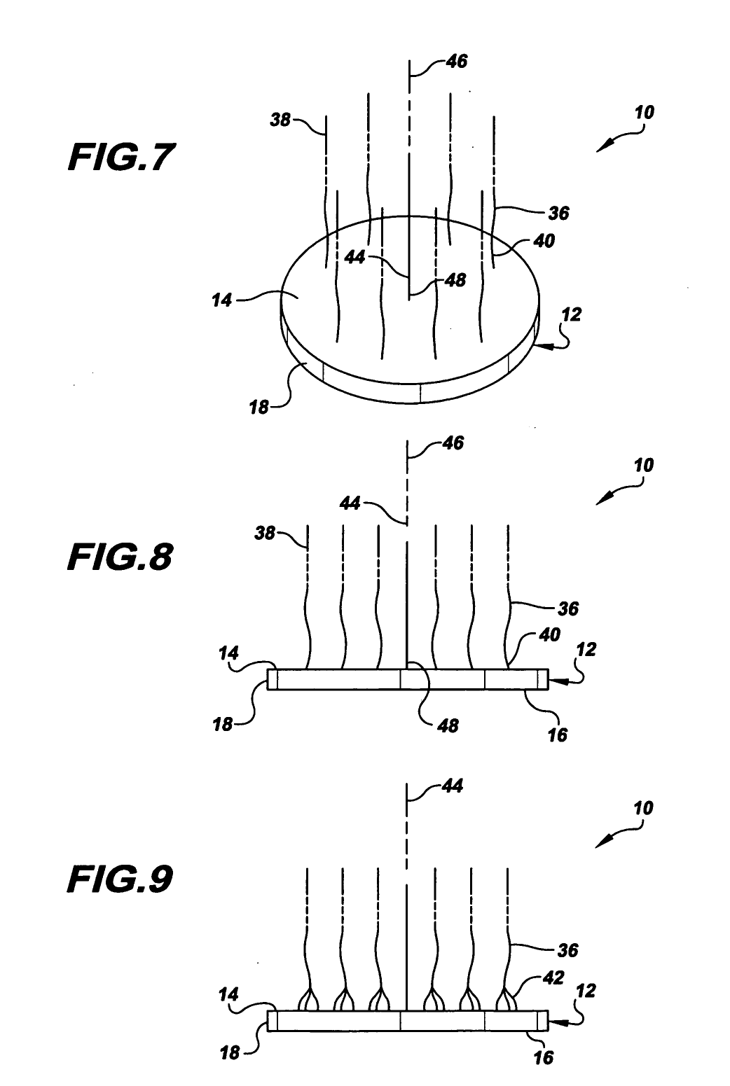

Referring now to FIG. 7, FIG. 8 and FIG. 9, there are shown a partial perspective view of the first side 14 of another embodiment of the device 10 for the obliteration of an aberrant space or cavity (FIG. 7); a partial lateral perspective view of the embodiment of the device 10 shown in FIG. 7 (FIG. 8); and a partial lateral perspective view of another embodiment of the device 10 (FIG. 9). The embodiment of the device 10 shown in FIG. 7 and FIG. 8 is particularly useful for obliterating an aberrant space or cavity, such as the hernia sac of an inguinal hernia, that is shallow and that has a particularly wide neck entry site into the space or cavity. As can be seen in FIG. 7 and FIG. 8, the device 10 can have the same components as the devices disclosed above, but in addition the device 10 further comprises a plurality of peripheral tethering sutures 36, each peripheral tethering suture 36 comprising a free first end 38, and comprising a second end 40 joined to the first surface 14 of the device 10. In one embodiment, the plurality of peripheral tethering sutures 36 is between 2 and about 10 tethering sutures 36. In a particularly preferred embodiment, the plurality of peripheral tethering sutures 36 is between about 4 and about 6 tethering sutures 36. In one embodiment, as seen in FIG. 9, the second end 40 of one or more than one of the peripheral tethering sutures 36 comprises a plurality of secondary sutures 42 joining the second end 40 of the peripheral tethering sutures 36 to the first side 14 of the device 10. In a preferred embodiment, the plurality of the secondary sutures 42 joining the second end of the peripheral tethering sutures 36 to the first side 14 comprises between 2 and about 10 secondary sutures 42. In a preferred embodiment, the peripheral tethering sutures 36 are arranged circumferentially around the center point of the first side 14. In a preferred embodiment, as shown in FIG. 7 and FIG. 8, the device 10 further comprises either a central tethering suture or a central stabilization wire 44 comprising a free first end 46 and a second end 48 joined to the first side 14 of the device 10. Preferably, the second end 48 of the central tethering suture or the central stabilization wire 44 joins the first side 14 of the device 10 approximately in the center point of the first side 14 and with the peripheral tethering sutures 36 positioned circumferentially around the central tethering suture or the central stabilization wire 44. In one embodiment, each tethering suture 36 comprises strong, nonresorbable surgical suture, such as, for example, braided polyester coated with PTFE (for example, Tevdek® suture, Genzyme Corporation Framingham, Mass., US), or nylon or silk suture material. In one embodiment, the stabilization wire comprises a suitable metal alloy such as stainless steel or titanium, or a rigid polymer with suitable shape memory. The stabilization wire can be flexible. In a preferred embodiment, the stabilization wire is sufficiently rigid to impart steerability or torquability to the device, and to permit back tension to be exerted on the device during manipulation. In one embodiment, the second end 48 of the central tethering suture or the central stabilization wire 44 is configured to detach from the device 10 by any of a variety of means, such as, for example, by a hydraulic, electrolytic, electrothermal or mechanical means, as will be understood by those with skill in the art with reference to this disclosure.

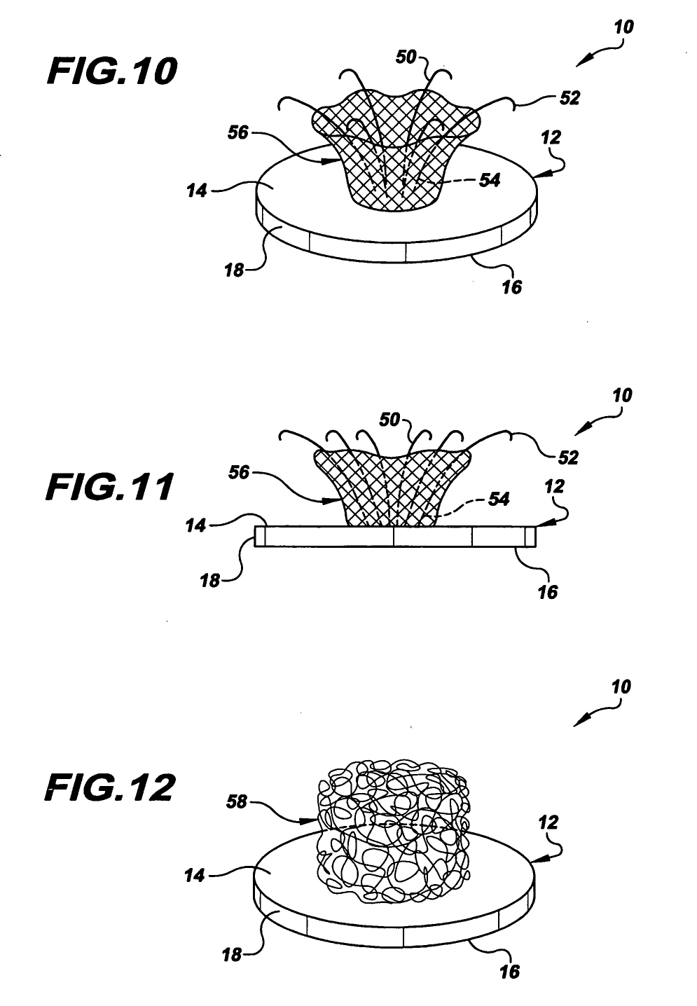

Referring now to FIG. 10 and FIG. 11, there are shown, respectively, a perspective view of the first side 14 of another embodiment of the device 10 for the obliteration of an aberrant space or cavity (FIG. 10); and a lateral perspective view of the embodiment of the device 10 shown in FIG. 10 (FIG. 11). Like the device 10 shown in FIG. 7 and FIG. 8, the embodiment of the device 10 shown in FIG. 10 and FIG. 11 is also particularly useful for obliterating an aberrant space or cavity, such as the hernia sac of an inguinal hernia, that is shallow and that has a particularly wide neck entry site into the space or cavity. As can be seen in FIG. 10 and FIG. 11, the device 10 can have the same components as the devices 10 disclosed above, but in addition the device 10 further comprises a plurality of wires 50, each wire 50 comprising a free first end 52, and a second end 54 joined to the first side 14 of the device 10. In one embodiment, the plurality of wires 50 is between about 2 and about 10 wires. In a particularly preferred embodiment, the plurality of wires 50 is between about 4 and about 6 wires. In a preferred embodiment, wire 50 comprises a suitable metal alloy such as stainless steel, titanium, nitinol or Elgiloy®, or a rigid polymer with suitable shape memory. When the device 10 is used to obliterate the hernia sac of an inguinal hernia, each wire 50 has an axial length of between about 0.5 cm and 2.0 cm, and a diameter between about 0.15 mm and 0.35 mm. In a preferred embodiment, the first end 52 of each wire bends between approximately 100° and 180° as shown in FIG. 10 and FIG. 11, and comprises a sharp tip, such as, for example, a barb, directed toward the first side 14 of the device 10. The sharp tips join the device 10 to a surface or structure adjacent to the aberrant space or cavity to be obliterated, such as to the peritoneal surface at the entry site into the hernia sac of an inguinal hernia, by penetrating into the peritoneal surface to a depth of approximately 1 mm, thereby immobilizing the device 10 at its deployment site. The bent portions of the wires 50, however, assist in preventing the wires 50 from penetrating into the tissue proximal to the deployment site. In a preferred embodiment, as shown in FIG. 10 and FIG. 11, the device 10 further comprises a material skirt 56 surrounding the second ends 54 of the wires 50 to promote a fibrotic reaction, and extending toward the first free ends 52 of the wires 50, while not covering the first free ends 52 of the wires 50. In a particularly preferred embodiment, such as when the device 10 is being used to close the hernia sac of an inguinal hernia, the material is biocompatible. In another preferred embodiment, the skirt 56 comprises a mesh of polypropylene or polyglycolic acid and extending toward the first free ends 52 of the wires 50, though other suitable materials can be used, as will be understood by those with skill in the art with reference to this disclosure.

Referring now to FIG. 12, there is shown a lateral perspective view of the first side 14 of another embodiment of the device 10 for the obliteration of an aberrant space or cavity. The device 10 comprises a mass of material 58 attached to the first side 14 of the device 10. The mass of material 58 can be incorporated into any of the embodiments of the device 10 according to the present invention. For illustrative purposes only, the mass of material 58 is shown in FIG. 12 incorporated into the device 10 shown in FIG. 1 and FIG. 2. The mass of material 58 comprises one or more than one substance or composition known to promote a fibrotic reaction in a human, such as polypropylene mesh or polyglycolic acid. When a device 10 according to the present invention, comprising the mass of material 58, is used for the obliteration of the hernia sac of an inguinal hernia, the mass of material 58 promotes a fibrotic reaction within the hernia sac, thereby assisting in closing the hernia sac.

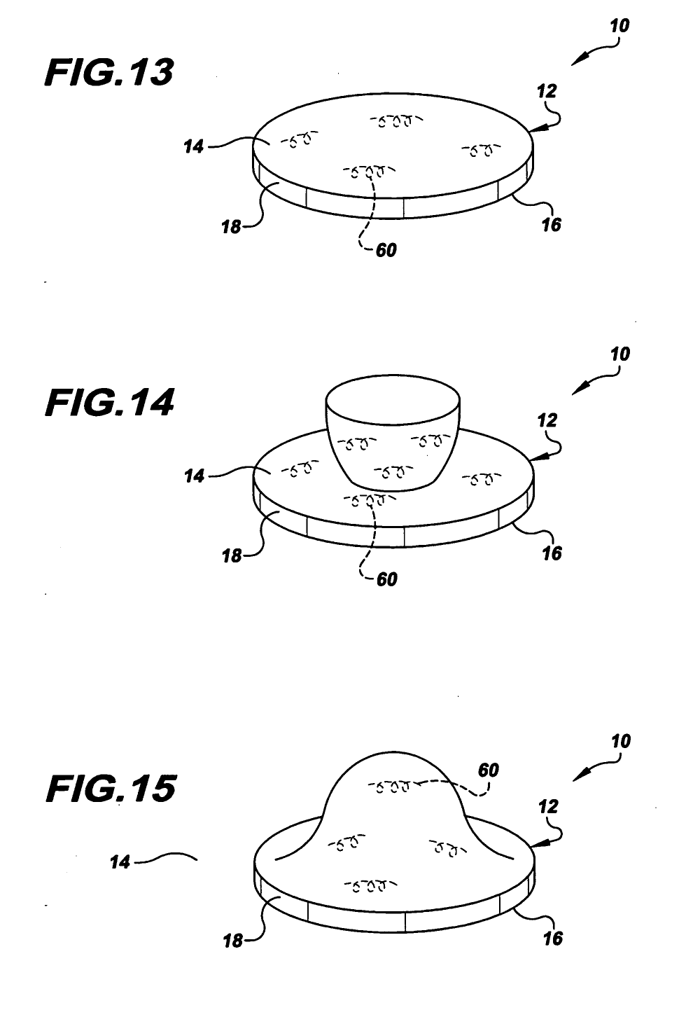

Referring now to FIG. 13, FIG. 14 and FIG. 15, there are shown lateral perspective views of three other embodiments of the device 10 for the obliteration of an aberrant space or cavity, consisting essentially of biocompatible, elastic memory foam and one or more than one resistive heating element 60. In one embodiment, the device 10 for the obliteration of an aberrant space or cavity comprises a disk 12, with or without additional structures, comprising a biocompatible, elastic memory foam whose final shape is attained after the application of heat provided by one or more than one resistive heating element 60 embedded within the disk 12, and other structures when present. In a preferred embodiment, the foam is a cold hibernated elastic memory (CHEM), polyurethane-based foam, such as used for endovascular interventions, or is a shaped memory polymer, such as a suitable polystyrene material, as will be understood by one of ordinary skill in the art with reference to this disclosure. The entire device 10 can consist of, or consist essentially of biocompatible, elastic memory foam and one or more than one resistive heating element 60 embedded within the disk 12, and within the other structures when present, as shown in FIG. 13, FIG. 14 and FIG. 15, or the device 10 can comprise additional structures according to other embodiments of the present invention, the additional structures comprising biocompatible, elastic memory foam and one or more than one resistive heating element 60, or not comprising biocompatible, elastic memory foam and one or more than one resistive heating element 60.

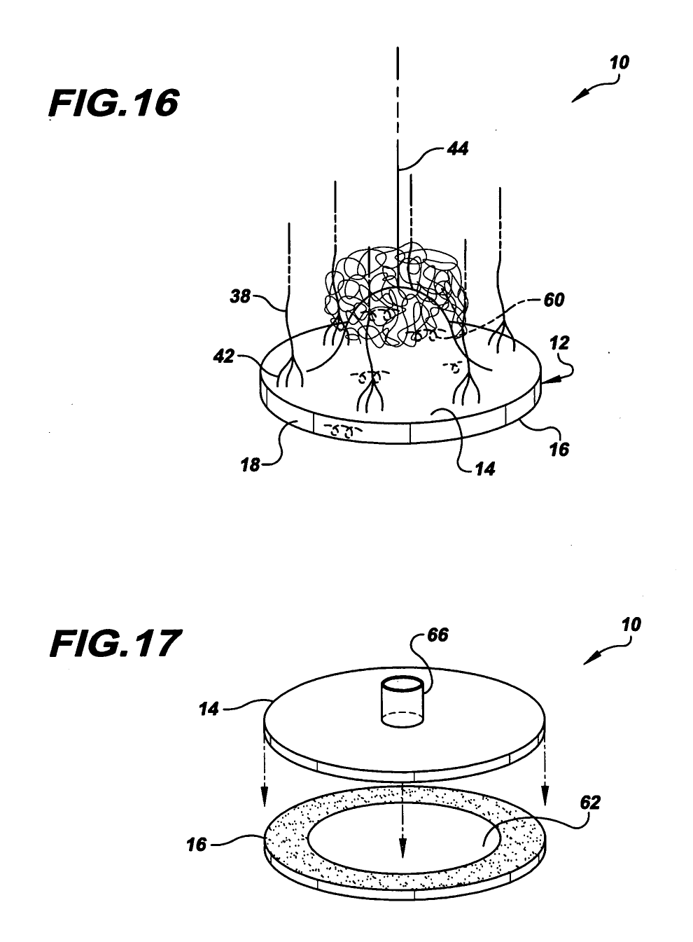

For example, referring now to FIG. 16, there is shown a lateral perspective view of the first side 14 of another embodiment of the device 10 for the obliteration of an aberrant space or cavity. This device 10 comprises the embodiment shown in FIG. 15, with the addition of tethering sutures 36 and a central stabilization wire 44, as shown in the embodiment in FIG. 7, FIG. 8 and FIG. 9, and as disclosed in reference to the embodiment in FIG. 7, FIG. 8 and FIG. 9. Additionally, the first side 14 of the embodiment shown in FIG. 16 is covered, partially or totally, with a material which promotes a fibrotic reaction in a human, such as polypropylene or polyglycolic acid. Further, the second side 16 of the embodiment shown in FIG. 16 is covered, partially or totally, with a material which inhibits a fibrotic reaction in a human, such as a material selected from the group consisting of a hydrophilic material, a biocompatible hydrogel, PTFE and a sodium hyaluronate and carboxy-methylcellulose-based material such as Seprafilm®. When the device 10 shown in FIG. 16 is used in a human to repair an inguinal hernia, the first side 14 with the material which promotes a fibrotic reaction in a human is positioned facing the hernia sac, and the second side 16 is positioned facing the intraperitoneal contents with the material which inhibits a fibrotic reaction in a human.

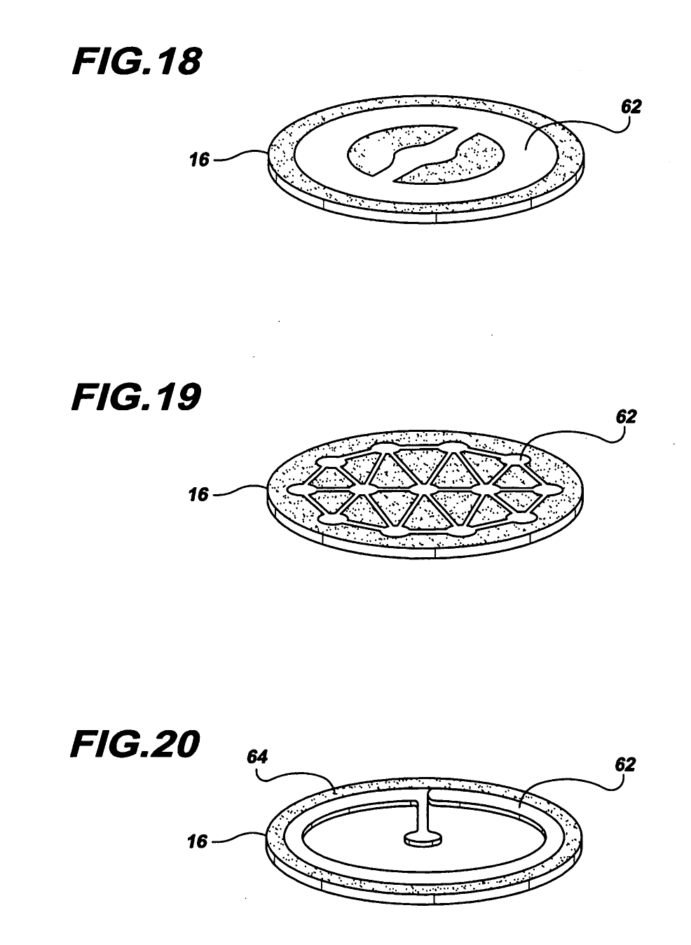

Referring now to, FIG. 17, FIG. 18, FIG. 19 and FIG. 20, there are shown, respectively, an exploded, lateral perspective view of the disk 12 portion of another embodiment of the device 10 for the obliteration of an aberrant space or cavity (FIG. 17); and top perspective views of the second side 16 of three embodiments of the device 10 for the obliteration of an aberrant space or cavity according to the present invention (FIG. 18, FIG. 19 and FIG. 20). As can be seen, these embodiments comprise one or more than one inflation area or inflation channel 62 between the first side 14 and the second side 16. FIG. 17 through FIG. 20, show some of the possible patterns in which the suitable adhesive can be applied; however, many other patterns are also possible, as will be understood by those with skill in the art with reference to this disclosure. The inflation area or inflation channel 62 can be made by, for example, applying a suitable adhesive in a variety of patterns to either the first side 14, the second side 16 or both the first side 14 and the second side 16, and joining the first side 14 to the second side 16, as shown in FIG. 18. In a preferred embodiment, the adhesive is applied around the outer edges of the first side 14, the second side 16 or both the first side 14 and the second side 16, to seal the edges of the first side 14 to the second side 16, in addition to being applied at any other location. Further, the inflation area or inflation channel 62 can be made by methods other than applying an adhesive, such as, for example, joining two layers of silicon or fluorosilicone rubber by the application of heat to create cavities between the otherwise sealed areas, where the cavities are in communication with each other, as shown in FIG. 19. Additionally, the inflation area or inflation channel 62 can be made by attaching a layer of material on either side of a substantially circular inflatable, polyethylene ring 64, as shown in FIG. 20. In a preferred embodiment, the device 10 further comprises a connector 66 in communication with the one or more than one inflation area or inflation channel 62 for interfacing with an inflation mechanism (not shown). The inflation mechanism connects to the connector 66, thereby allowing a gas, air, liquid or another suitable inflation medium to enter the inflation area or inflation channel 62 through the inflation mechanism, and thereby separating the first side 14 from the second side 16 where the first side 14 and the second side 16 are not joined directly, and thereby increasing the effective thickness and stiffness of the disk 12 of the device 10. This embodiment permits the deployment of the device 10 while it has a smaller profile, prior to inflation. In a preferred embodiment, the connector 66 is a one-way valve which permits inflation of the inflation area or inflation channel 62, and the connector 66 is then self-sealing.

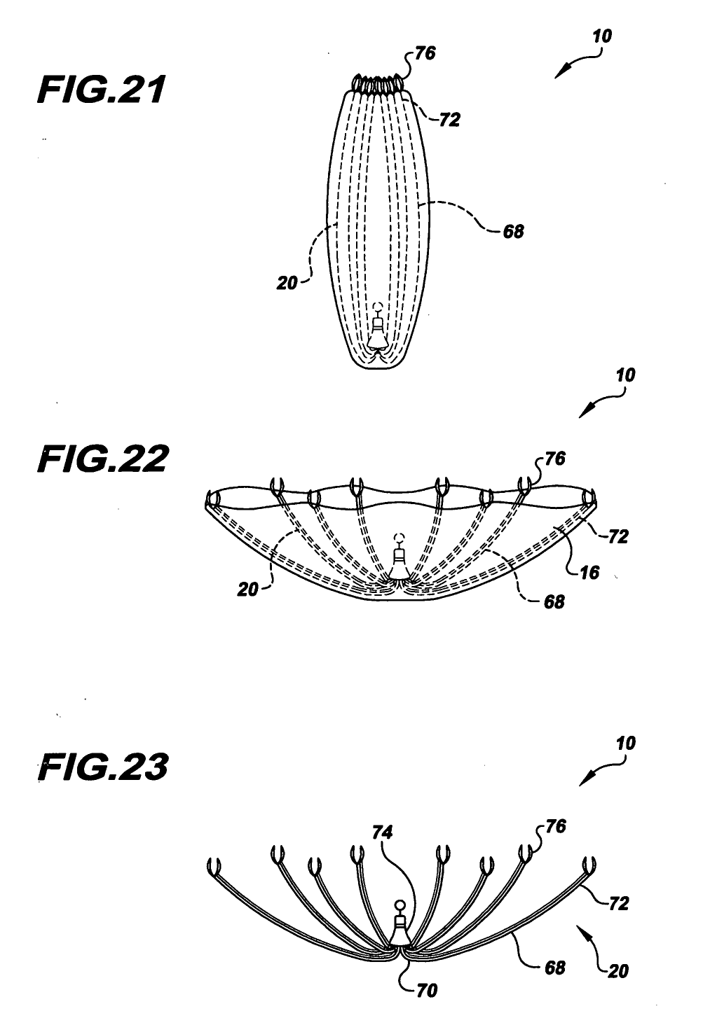

Referring now to FIG. 21, FIG. 22 and FIG. 23, there are shown, respectively, a lateral perspective view of another embodiment of the device 10 for the obliteration of an aberrant space or cavity in a pre-deployment configuration (FIG. 21); a lateral perspective view of the embodiment of the device 10 shown in FIG. 21 in a post-deployment configuration (FIG. 22); and a lateral perspective view of the frame 20 portion of the device 10 shown in FIG. 22 in a post-deployment configuration (FIG. 23). As can be seen, in this embodiment, the device 10 comprises a first side 14, an opposing second side 16, and a frame 20 between the first side 14 and the second side 16. The first side 14 and the second side 16 can comprise any of the materials disclosed in this disclosure for the first side 14 and the second side 16 of the device 10. In this embodiment, the frame 20 comprises a plurality of peripherally radiating members 68 comprising a first end 70 and a second end 72. The first end 70 of each radiating member is joined at a central connector 74. The second end 72 of one or more than one of the radiating members 68 comprises a clip 76 to attach the radiating member 68 to a surface or structure adjacent to or within the aberrant space or cavity to be obliterated, such as to the peritoneal surface at the entry site into the hernia sac of an inguinal hernia, thereby immobilizing the device 10 in position. As can be seen with particular reference to FIG. 21, when this embodiment in a pre-deployment configuration, the second ends 72 of the radiating members 68 approximate, thereby rendering the device 10 into a smaller profile suitable for deployment through a small opening. As can be seen with particular reference to FIG. 22 and FIG. 23, after deployment, the second ends 72 of the radiating members 68 are separated by actuating a mechanism in the central connector 74, thereby rendering the device 10 into a post-deployment configuration suitable for sealing the opening of the aberrant space or cavity. In one embodiment, the frame 20 comprises wire, such as a shaped metal alloy or is a shaped memory polymer, such as a suitable polystyrene material. In a preferred embodiment, the metal alloy is selected from the group consisting of a nitinol and Elgiloy®. In a preferred embodiment, when the device 10 is used to obliterate an aberrant space or cavity within a human, the frame 20 comprises wire of a biocompatible material.

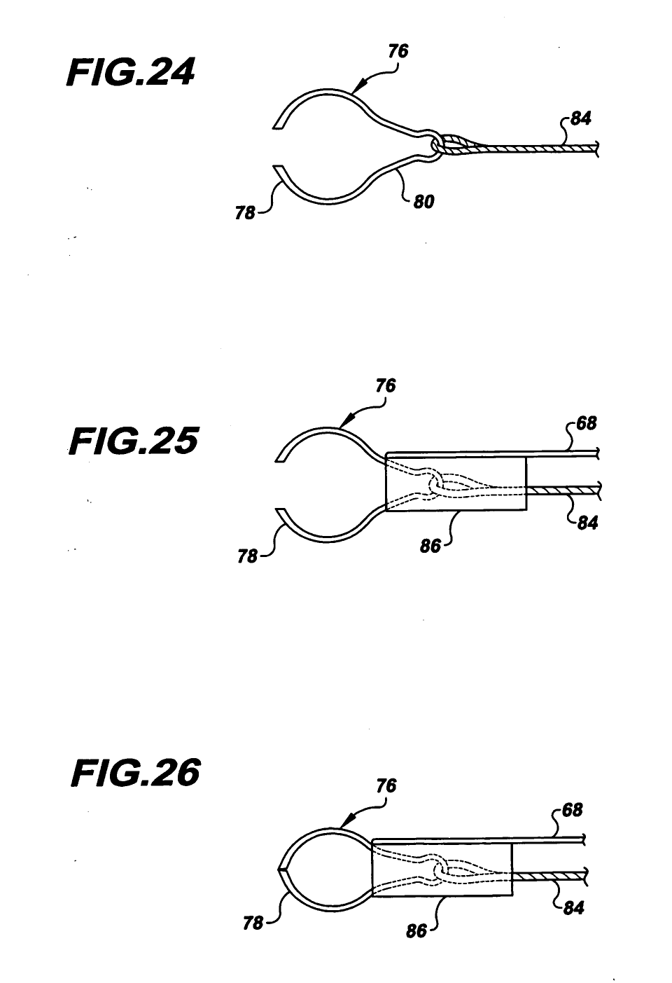

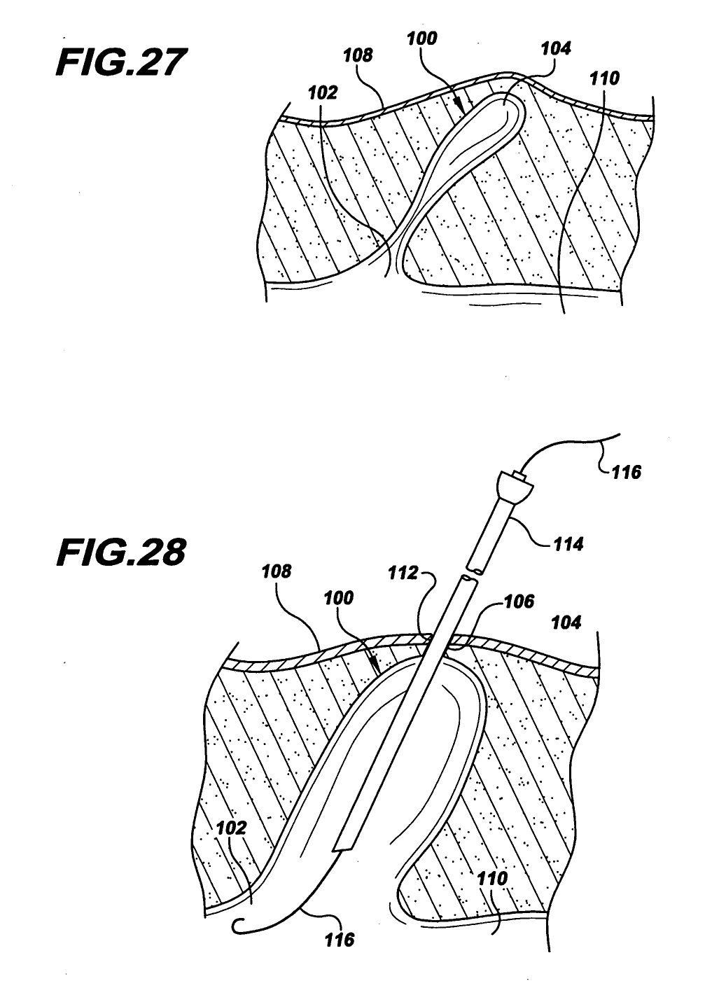

Referring now to FIG. 24, FIG. 25 and FIG. 26, there are shown, respectively, a close-up, lateral perspective views of parts of one embodiment of a clip 76 suitable for incorporation into the embodiment shown in FIG. 21, FIG. 22 and FIG. 23. As will be understood by those with skill in the art with reference to this disclosure, other clips are also suitable for incorporation into the device 10. In the embodiment shown in FIG. 24, FIG. 25 and FIG. 26, the clip 76 comprises a plurality of arms 78 comprising a first end 80 and a second end 82. The first ends 80 of each arm 78 are joined to an attachment line 84. In a preferred embodiment, the second end 82 of each arm 78 comprises one or more than one gripping tip, such as, for example, a sharp point or barb. Though shown with only two arms 78, each clip 76 can comprise three or more arms, as will be understood by those with skill in the art with reference to this disclosure. Each clip 76 further comprises a tubular structure 86 surrounding the first end 76 and attached to the frame 20. As can be seen with particular reference to FIG. 25, in the pre-deployment position, the second ends 82 of each arm 78 are separated from each other and extend maximally outside the tubular structure 86. As can be seen with particular reference to FIG. 26, after deployment, axial force is applied to the attachment line 84, toward the central connector 74 of the frame 20, thereby translating the joined first ends 80 of the clip 76 axially toward the central connector 74, and approximating the second ends 82 of the clip 76. Any surface or structure adjacent to or within the aberrant space or cavity to be obliterated between the second ends 82 of the clip 76 at the time of deployment is then caught between the second ends 82, thereby immobilizing the device 10 in position.

According to another embodiment of the present invention, there is provided a method for the obliteration of an aberrant space or cavity comprising an open end and a closed end. In a preferred embodiment, the aberrant space or cavity obliterated by the method is within a living organism, such as within a human. In another preferred embodiment, the aberrant space or cavity obliterated by the method is a hernia sac of an inguinal hernia. In one embodiment, the method comprises providing a device according to the present invention, and deploying the device to substantially seal the open end of the aberrant space or cavity. In another embodiment, the method comprises creating an opening in the closed end of the aberrant space or cavity, introducing a device through the opening in the closed end of the aberrant space or cavity, and deploying the device to substantially seal the open end of the aberrant space or cavity. In a preferred embodiment, the device introduced is a device according to the present invention.

By way of example, the method will now be disclosed in greater detail with specific reference to the obliteration of a hernia sac of an inguinal hernia. As will be understood by those with skill in the art with reference to this disclosure, however, equivalent steps can be used to obliterate any aberrant space or cavity suitable for obliteration by the present method, including an aberrant space or cavity other than the hernia sac of an inguinal hernia, other than within a living organism, and other than within a human.

As will be appreciated by one with skill in the art with reference to this disclosure, when the method of the present invention is used to obliterate the hernia sac of an inguinal hernia, the method preferably involves a percutaneous transcatheter approach, though a laparoscopic approach or open surgical approach can also be used. Specifically, in a preferred embodiment, the method comprises deploying a device through an opening created in the closed end of the aberrant space or cavity using a percutaneous transcatheter approach. Compared with open surgical and laparoscopic approaches for the treatment of inguinal hernias currently performed, the percutaneous transcatheter approach of the present method reduces procedure times, decreases risks of infection, requires smaller incisions and fewer punctures, and reduces recuperation time. Further, the method reduces procedural costs due to the utilization of an interventional radiology suite for the repair instead of a more expensive operating room environment.

Referring now to FIG. 27 through FIG. 36, there are shown, a partial cross-sectional view of an aberrant space or cavity 100 comprising an open end 102 and a closed end 104, suitable for obliteration according to a method of the present invention (FIG. 27); and partial cross-sectional views of various steps of some embodiments of the method of the present invention for the obliteration of an aberrant space or cavity 100 comprising an open end 102 and a closed end 104 (FIG. 28 through FIG. 36). The steps shown are not intended to be limiting nor are they intended to indicate that each step depicted is essential to the method, but instead are exemplary steps only.

The method comprises, first selecting an aberrant space or cavity 100 that is suitable for obliteration by the method. The aberrant space or cavity 100 comprises an open end 102 and a closed end 104. In one embodiment, the aberrant space or cavity 100 is within a living organism. In a preferred embodiment, the aberrant space or cavity 100 is within a human. In a particularly preferred embodiment, the aberrant space or cavity 100 is a hernia sac of an inguinal hernia within a human.

In one embodiment, selecting an aberrant space or cavity 100 that is suitable for obliteration by the method comprises selecting a patient having a disease or condition that includes the existence of an aberrant space or cavity 100, such as, for example, the hernia sac of an inguinal hernia, and for which treatment of the disease or condition involves obliteration of the aberrant space or cavity 100. In this embodiment, selecting the patient can comprise diagnosing the existence of an aberrant space or cavity 100 using standard techniques, such as a technique selected from the group consisting of CT scan, herniography, history, MRI and physical examination.

The following steps are disclosed with respect to obliterating the hernia sac 100 of an inguinal hernia as an example. Next, anesthesia is induced, and the lower abdominal and inguinal areas prepped and draped in a sterile fashion, according to standard techniques. Then, an opening 106 is created in the closed end 104 of the hernia sac 100. A device for obliterating the hernia sac 100 is deployed, thereby obliterating the hernia sac 100. In a preferred embodiment, the device deployed is a device 10 for the obliteration of an aberrant space or cavity 100 according to the present invention. In another preferred embodiment, the device is introduced percutaneously. Introduction of the device can be accomplished in a number of ways depending on the embodiment of the device used, as will be understood by those with skill in the art with reference to this disclosure.

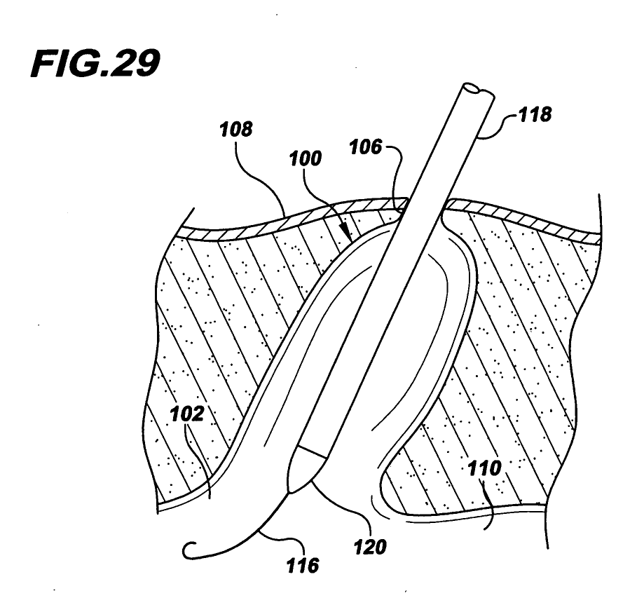

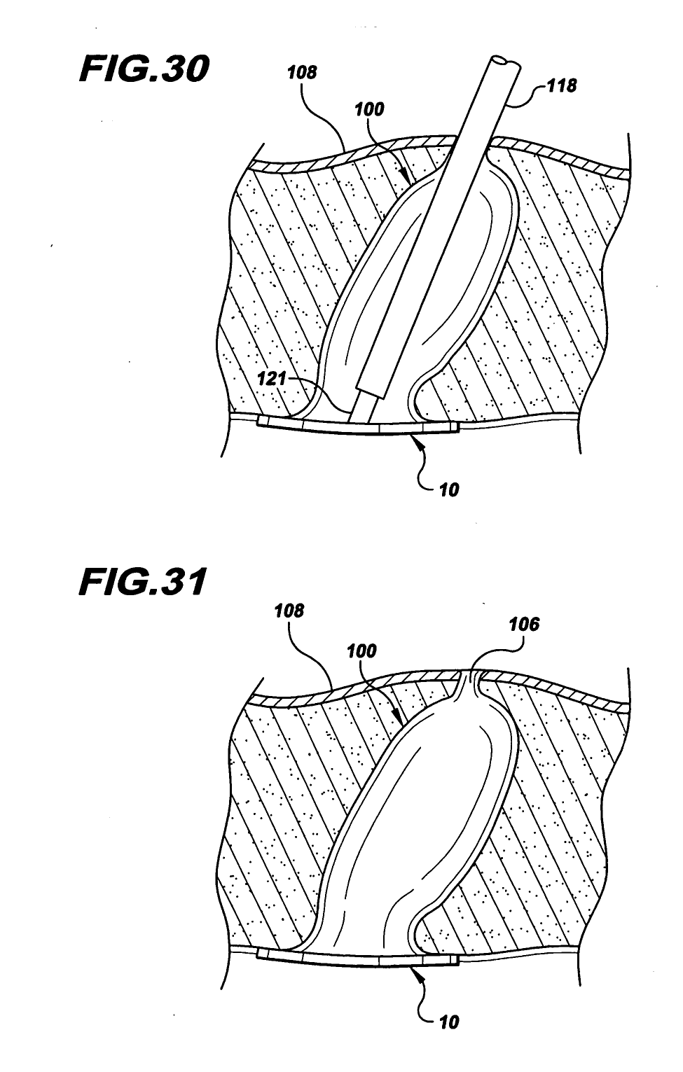

By way of example only, various introduction steps will now be disclosed in detail. A puncture incision is made in the skin of the lower abdomen 108 with a 20-22 gauge needle. In one embodiment, the peritoneal cavity 110 is inflated with a suitable gas, such as, for example, carbon dioxide gas, which also distends the hernia sac 100. As can be seen in FIG. 28, the closed end 104 of the distended hernia sac 100 is then entered by a second incision 112, such as a puncture incision, with an 18-gauge needle 114 creating an opening 106 in the closed end 104. A 1 mm diameter guidewire 116 is advanced under suitable guidance, such as, for example, fluoroscopic guidance, through the opening 106 in the closed end 104 of the hernia sac 100, through the hernia sac 100, and through the open end 102 of the hernia sac 100 into the peritoneal cavity 110. Next, as can be seen in FIG. 29, the needle 114 is removed, and over the guidewire 116, a 12-14 F introducer catheter 118 with its central dilator 120 is advanced through the opening 106 in the closed end 104 of the hernia sac 100, through the hernia sac 100, and through the open end 102 of the hernia sac 100 into the peritoneal cavity 110. The guidewire 116 and central dilator 120 are then removed.

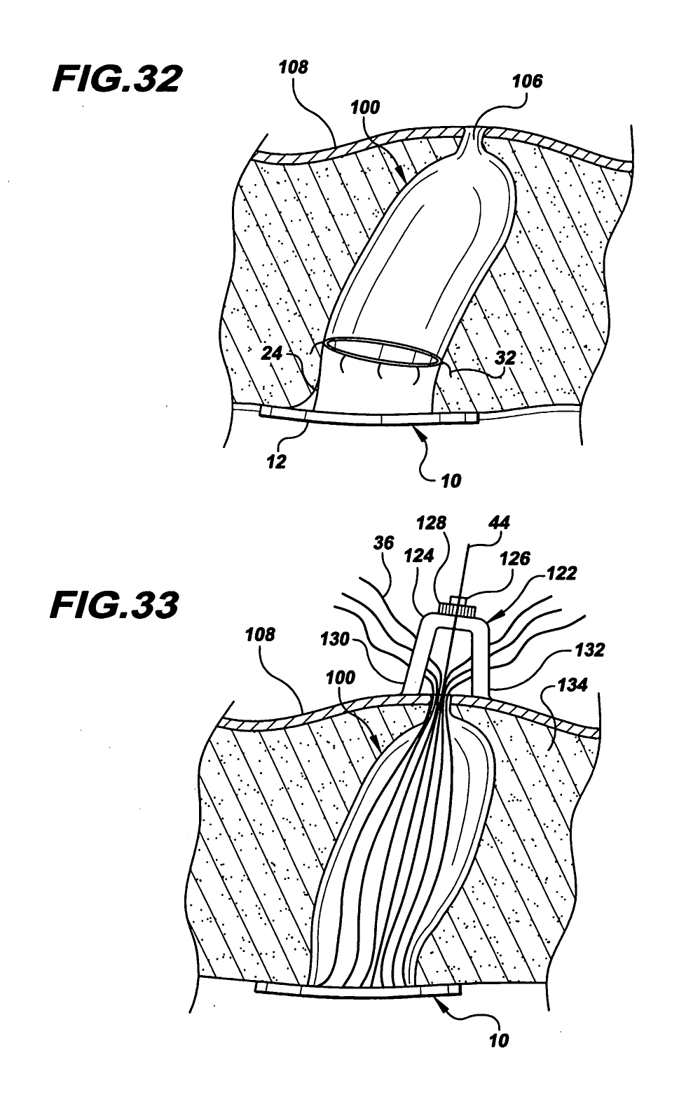

Next, a device 10 according to the present invention in its pre-deployment configuration is advanced into the hernia sac 100. In a preferred embodiment, the device 10 is advanced through the introducer catheter 118 directly. Introducing the device 10 into the hernia sac 100, whether through the introducer catheter 118 or not, can comprise collapsing the device 10 by bringing the perimeter 18 of the device 10 toward the center, by rolling the device 10, or by another method as will be understood by those with skill in the art with reference to this disclosure. As can be seen in FIG. 30 and FIG. 31, the device 10 is deployed at or near the open end 102 of the hernia sac 100 at the junction of the peritoneal cavity 110. As appropriate for the embodiment of the device 10, the device 10 can be attached to a pusher rod 121 to assist in proper placement of the device 10 during deployment. The deployed device 10 is shown in FIG. 30 and FIG. 31 as a disk 12 only, which is intended to schematically indicate the deployment of the disk 12 portion of any of the embodiments of the devices 10 according to the present invention, or any other suitable device as will be understood by those with skill in the art with reference to this disclosure.

FIG. 32 through FIG. 36 show deployment and immobilization of various embodiments of the device 10 of the present invention. FIG. 32 shows the deployment position of the device 10 shown in FIG. 5 and FIG. 6. As can be seen, when deployed, the connectors 32 on the first end 52 of the main section 26 join the device 10 to the peritoneal surface adjacent to or within the hernia sac 100, thereby immobilizing the device 10 in position. In one embodiment of the method, deployment is accomplished by extruding the disk 12 of the device 10 from the introducer catheter 118 under fluoroscopic guidance until the disk 12 is fully expanded within the gas-distended peritoneal cavity 110. Further under fluoroscopic guidance, the introducer catheter 118 with the exposed expanded disk 12 is pulled back until slight resistance is encountered as the disk 12 meets the peritoneal surface at the junction of the open end 102 of the hernia sac 100 and the peritoneal cavity 110, due to the diameter of the disk 12 being greater than the diameter of the open end 102 of the hernia sac 100. Slight back traction is maintained on the device 10, if necessary, by pulling on an attached pusher rod. While the back traction is maintained, the introducer catheter 118 is slowly withdrawn allowing the main section 26 to gradually expand until the connectors 32 expand sufficiently radially outward to join the device 10 to the peritoneal surface adjacent to or within the hernia sac 100, thereby immobilizing the device 10 in position. The back traction on the pusher rod is relaxed, and fluoroscopy is used to ensure that the device 10 is properly positioned. Once the device 10 is properly placed, the pusher rod is detached from the device 10 using standard techniques such as, for example, by a hydraulic, electrolytic, electrothermal or mechanical method. The introducer catheter 118 is then removed and the incision 112, and any other incision, is closed according to standard techniques.

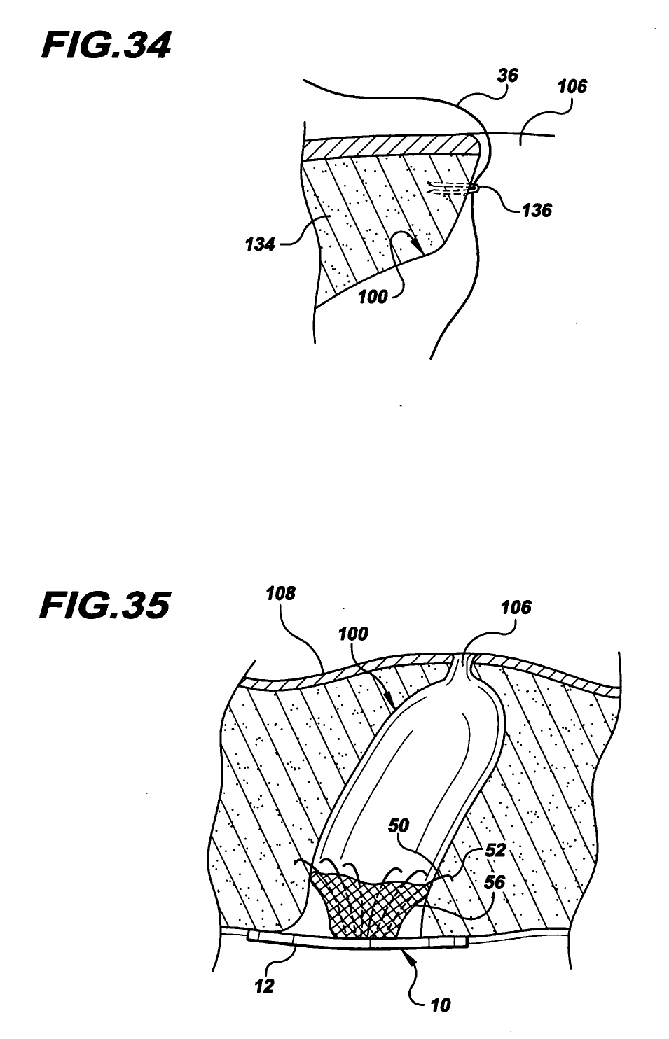

FIG. 33 and FIG. 34 shows two steps in the method of the present invention using the device 10 shown in FIG. 7 and FIG. 8. As can be seen in FIG. 33, when the device 10 is deployed the first free ends 38 of the plurality of peripheral tethering sutures 36 extend from the first side 14 of the disk 12 through the hernia sac 100, out of the opening 106 in the closed end 104 of the hernia sac 100, and out of the incision 112 in the skin above the hernia sac 100. The tethering sutures 36 are fixed to the tissue over the hernia sac 100 by, for example, extending the first free ends 38 through separate subcutaneous tunnels and then tying the first free ends 38 together, or by another method as will be understood by those with skill in the art with reference to this disclosure. When present, the central stabilization wire or central suture 44 also extends from the first side 14 of the disk 12 through the hernia sac 100, out of the opening 106 in the closed end 104 of the hernia sac 100, and out of the incision 112 in the skin above the hernia sac 100. In a preferred embodiment, back traction is maintained on the central tethering suture or a central stabilization wire 44 after withdrawing the introducer catheter 118 by a traction device 122. In one embodiment, as shown in FIG. 33, the traction device 122 comprises a first end 124 having a port 126 to receive the central tethering suture or a central stabilization wire 44, and a mechanism 128 to apply tension to the central tethering suture or a central stabilization wire 44. The traction device 122 further comprises a second end 130 comprising a plurality of legs 132. As shown in FIG. 34, once back traction is achieved, the first free ends 38 of the plurality of peripheral tethering sutures 36 are fixed into the subcutaneous tissues 134, such as, for example, by using one or more than one small biocompatible metallic or plastic tissue clips 136 to anchor one or more than one of the first free ends 38 of the plurality of peripheral tethering sutures 36, thereby immobilizing the device 10 in position. Any excess suture proximal to the clip 136 is then removed below the skin surface. Then, the second end 130 of the central tethering suture or a central stabilization wire 44 is detached from the device 10 by any of a variety of means, such as, for example, by a hydraulic, electrolytic, electrothermal or mechanical means, as will be understood by those with skill in the art with reference to this disclosure. Finally, the incision 112 is closed according to standard techniques. As will be understood by those with skill in the art with reference to this disclosure, the embodiment of the device 10 shown in FIG. 9 is deployed using steps equivalent to the steps disclosed with respect to the embodiment shown in FIG. 7 and FIG. 8.

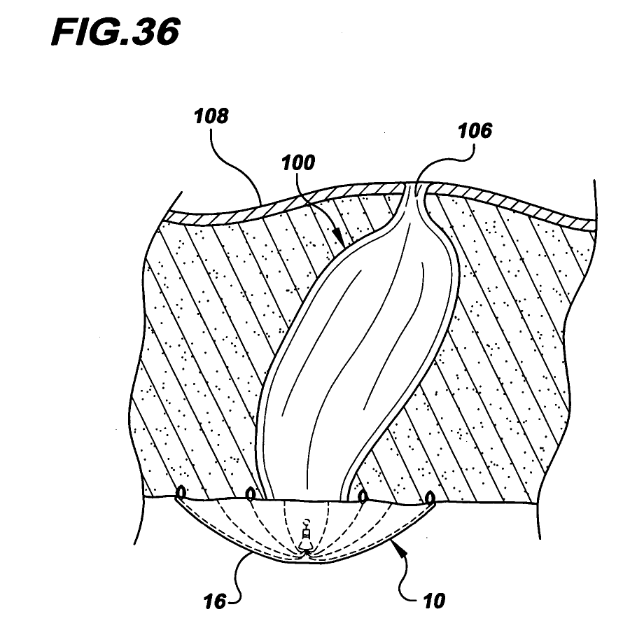

FIG. 35 shows the deployment position of the device 10 shown in FIG. 10 and FIG. 11. As can be seen, when deployed, the sharp tips on the first ends 52 of the wires 50 join the device 10 to the peritoneal surface adjacent to or within the hernia sac 100, thereby immobilizing the device 10 in position. In one embodiment of the method, deployment is accomplished using steps corresponding to the steps disclosed for the method in general, and with respect to the device 10 shown in FIG. 5 and FIG. 6 more specifically. In a preferred embodiment, the method further comprises attaching the pusher rod to the first ends 52 of the wires 50, such as, for example, using a snare or suture or equivalent structure, running through the bent sections of the first ends 52 of the wires 50. Tension on the snare or suture is released, allowing the first ends 52 of the wires 50 to expand sufficiently radially outward to join the device 10 to the peritoneal surface adjacent to or within the hernia sac 100. Preferably, the position of the device 10 is then determined using a standard technique, such as, for example, fluoroscopy, and if the position is not satisfactory, the device 10 can be repositioned by tightening snare or suture, thereby drawing together the first ends 52 of the wires 50 of the device 10, which allows the device 10 to be repositioned. Once the position of the device 10 is satisfactory, the first ends 52 of the wires 50 are allowed to expand sufficiently radially outward to join the device 10 to the peritoneal surface adjacent to or within the hernia sac 100, thereby immobilizing the device 10 in position, and the pusher rod is detached from the device 10 and removed from the hernia sac 100.

When the device 10 used in the present method comprises inflation area or inflation channel 62, as disclosed with respect to the devices 10 shown in FIG. 17, FIG. 18, FIG. 19 and FIG. 20, the method further comprises inflating the inflation area or inflation channel 62, as will be understood by those with skill in the art with reference to this disclosure.

FIG. 36 shows the deployment position of the device 10 shown in FIG. 21 through FIG. 26. As can be seen, when deployed, the clips 76 attach the frame 20, and hence the device 10, to the peritoneal surface adjacent to or within the hernia sac 100, thereby immobilizing the device 10.

In one embodiment of the method, a mass of material 58 comprising one or more than one substance or composition known to promote a fibrotic reaction in a human, such as polypropylene mesh, is attached to the first side 14 of the disk 12 prior to introduction of the device 10 into the hernia sac 100. In another embodiment of the method, a mass of material 58 comprising one or more than one substance or composition known to promote a fibrotic reaction in a human, such as polypropylene mesh, is introduced into the hernia sac 100 after deployment of the device 10 and before closing the skin incision 112. The mass of material 58 promotes a fibrotic reaction within the hernia sac 100 and between the device 10 and the peritoneum, thereby assisting in obliterating the hernia sac 100.

In each of the embodiments of the method according to the present invention, after the incision or incisions are closed, the walls of the hernia sac will tend to join together further due to a fibrotic reaction caused, in part, by the device. This reaction assists in preventing recurrence of the hernia.

Although the present invention has been discussed in considerable detail with reference to certain preferred embodiments, other embodiments are possible. Therefore, the scope of the appended claims should not be limited to the description of preferred embodiments contained in this disclosure. All references cited herein are incorporated by reference to their entirety.

What is claimed is:

1. A device for the obliteration of an aberrant space or cavity comprising a disk of material comprising a first side, an opposing second side, and a perimeter circumferentially surrounding the first side and the second side.

2. The device of claim 1, further comprising a frame forming the perimeter, where the material forming the first side and the second side are stretched over the frame and attached to the frame.

3. The device of claim 2, where the frame comprises a shape selected from the group consisting of substantially round, oval, square, rectangular, kidney shaped, and clover leaf shaped with a plurality of leaves.

4. The device of claim 1, further comprising a central layer of compressible material between the first side and the second side, and within the perimeter.

5. The device of claim 4, where the central layer comprises a biocompatible, elastic memory foam whose final shape is attained after the application of heat provided by one or more than one resistive heating element embedded within the central layer.

6. The device of claim 1, where the first side of the device, the second side of the device, or both the first side and the second side of the device are covered, at least in part, by a material that inhibits a fibrotic reaction in a human.

7. The device of claim 1, where the first side of the device, the second side of the device, or both the first side and the second side of the device are covered, at least in part, by a material that promotes a fibrotic reaction in a human.

8. The device of claim 1, further comprising one or more than one attachment portion that is configured to attach one side of the device to a surface or structure adjacent to or within the aberrant space or cavity to be obliterated thereby immobilizing the device in position.

9. The device of claim 8, where the attachment portion comprises a main section comprising a first end, and a second end joined to the first surface of the device.

10. The device of claim 9, where the main section comprises a self-expanding stent comprising a wire comprising a plurality of angles to form a cylindrical shape.

11. The device of claim 9, where the first end of the main section comprises a plurality of connectors configured to join the device to a surface or structure adjacent to or within the aberrant space or cavity to be obliterated.

12. The device of claim 1, further comprising a plurality of peripheral tethering sutures, each peripheral tethering suture comprising a free first end, and comprising a second end joined to the first surface of the device.

13. The device of claim 12, where the second end of the one or more than one peripheral tethering sutures comprises a plurality of secondary sutures joining the second end peripheral tethering suture to the first surface.

14. The device of claim 13, further comprising a central tethering suture or a central stabilization wire comprising a free first end, and a second end joined to the first surface of the device.

15. The device of claim 1, further comprising a plurality of wires, each wire comprising a free first end, and a second end joined to the first surface of the device;
where the first end of each wire bends between approximately 100° and 180° and comprises a sharp tip directed toward the first surface of the device.

16. The device of claim 15, further comprising a material skirt surrounding the second ends of the wires and extending toward the first free end of the wires, but not covering the first free ends of the wires.

17. The device of claim 1, further comprising a mass of material attached to the first surface of the device, where the mass of material comprises one or more than one substance or composition known to promote a fibrotic reaction in a human.

18. The device of claim 1, where the device comprises a cold hibernated elastic memory, polyurethane-based foam.

19. The device of claim 18, further comprising one or more than one resistive heating element.

20. The device of claim 1, consisting essentially of biocompatible, elastic memory foam and one or more than one resistive heating element embedded within the disk.

21. The device of claim 1, consisting of biocompatible, elastic memory foam and one or more than one resistive heating element embedded within the disk.

22. The device of claim 1, further comprising one or more than one inflation channel between the first side and the second side.

23. The device of claim 1, further comprising a frame;
where the frame comprises a plurality of peripherally radiating members comprising a first end and a second end;
where the first end of each radiating member is joined at a central connector; and
where the second end of one or more than one of the radiating members comprises a clip to attach the radiating member to a surface or structure adjacent to or within the aberrant space or cavity to be obliterated thereby immobilizing the device in position.

24. The device of claim 23, where each clip further comprises a tubular structure surrounding the first end and attached to the frame.

25. A method for the obliteration of an aberrant space or cavity comprising an open end and a closed end, the method comprising:
a) selecting an aberrant space or cavity that is suitable for obliteration by the method;
b) creating an opening in the closed end of the aberrant space or cavity;
c) providing a device for the obliteration of an aberrant space or cavity; and
d) deploying the device through the opening created in the closed end of the aberrant space or cavity to substantially seal the open end of the aberrant space or cavity, thereby obliterating the aberrant space or cavity.

26. The method of claim 25, where the aberrant space or cavity obliterated by the method is within a living organism.

27. The method of claim 25, where the aberrant space or cavity obliterated by the method is a hernia sac of an inguinal hernia in a patient, and where the open end of the hernia sac is in communication with the peritoneal cavity of the patient.
